SEQUENCE LISTING (12 sequences)

SEQ ID NO: 1 - DNA, 26 bp, Artificial Sequence
  Description of Artificial Sequence Synthetic primer
ctagaagctttgtgaaacagctgccc

SEQ ID NO: 2 - DNA, 27 bp, Artificial Sequence
  Description of Artificial Sequence Synthetic primer
cgctagtctagatgtctctcaaatttc

SEQ ID NO: 3 - DNA, 42 bp, Artificial Sequence
  Description of Artificial Sequence Synthetic primer
ctctgtctcaaatttgtggtccactgagctataacttacttc

SEQ ID NO: 4 - DNA, 42 bp, Artificial Sequence
  Description of Artificial Sequence Synthetic primer
gaagtaagttatagctcagtggaccacaaatttgagacagag

SEQ ID NO: 5 - DNA, 51 bp, Artificial Sequence
  Description of Artificial Sequence Synthetic oligonucleotide
tccgtctctgtctcaaatttgtggtscactgagctataacttacttctgta

SEQ ID NO: 6 - RNA, 22 nt, Artificial Sequence
  Description of Artificial Sequence Synthetic oligonucleotide
ucagugcacuacagaacuuugu

SEQ ID NO: 7 - RNA, 22 nt, Artificial Sequence
  Description of Artificial Sequence Synthetic oligonucleotide
ucagugcaucacagaacuuugu

SEQ ID NO: 8 - RNA, 22 nt, Artificial Sequence
  Description of Artificial Sequence Synthetic oligonucleotide
ucagugcaugacagaacuuggg

SEQ ID NO: 9 - RNA, 54 nt, Artificial Sequence
  Description of Artificial Sequence Synthetic oligonucleotide
cuccgucucugucucaaauuuguggugcacugucagugcacuacagaacuuugu

SEQ ID NO: 10 - RNA, 54 nt, Artificial Sequence
  Description of Artificial Sequence Synthetic oligonucleotide
cuccgucucugucucaaauuugugguccacugucagugcacuacagaacuuugu

SEQ ID NO: 11 - RNA, 21 nt, Homo sapiens
ucaaauuuguggugcacugag

SEQ ID NO: 12 - RNA, 21 nt, Homo sapiens
ucaaauuugugguccacugag

(19) United States
(12) Patent Application Publication
(10) Pub. No.: US 2009/0156532 A1
(43) Pub. Date: Jun. 18, 2009

(54) SNP BINDING SITE FOR microRNAs IN HLA-G

(75) Inventors: Carole Ober, Chicago, IL (US); Zheng Tan, Chicago, IL (US); Jihua Fan, New York, NY (US)

Correspondence Address: BARNES & THORNBURG LLP, P.O. BOX 2786, CHICAGO, IL, 60690-2786, US

(73) Assignee: THE UNIVERSITY OF CHICAGO, Chicago, IL (US)

(21) Appl. No.: 12/160,930

(22) PCT Filed: Jan. 23, 2007
(86) PCT No.: PCT/US07/60928

§ 371 Date: Nov. 26, 2008

Related U.S. Application Data
(60) Provisional application No. 60/761,488, filed on Jan. 24, 2006.
(60) Provisional application No. 60/791,579, filed on Apr. 11, 2006.
(60) Provisional application No. 60/747,630, filed on May 18, 2006.

(51) Int. Cl. A61K 31/7105; C12Q 1/68

(52) U.S. Cl. 514/44; 435/6

(57) ABSTRACT

Analysis of microRNA interference with HLA-G expression identified the HLA-G 3′UTR SNP+3142G/C that disrupts a target for microRNA 148 (miR148) and is associated with asthma. The polymorphism is associated with protection from (or susceptibility to) moderate to severe viral infection in the first 3 years of life and asthma by age 6, with an interaction with mother's affection status (asthma). A SNP in the 3′-Untranslated Region (UTR) of HLA-G influences the targeting of 3 micro(mi)RNAs to the gene. Allele-specific targeting of these miRNAs accounts, at least in part, for the association between HLA-G and the risk of asthma.

### BACKGROUND

HLA-G 3′UTR is a target site for microRNAs miR-148a, -148b, and -152. The effects of allelic variation at position +3142 C/G on miRNA targeting are demonstrated. Associations between +3142 C/G and asthma-related phenotypes are evaluated.

The HLA region on the short arm of chromosome 6 (6p21.3) contains the most polymorphic coding sequences in the human genome. HLA allele frequency distributions as well as patterns of linkage disequilibrium differ significantly among different human populations. HLA population data can be valuable in estimating the likelihood of finding an HLA-matched donor for bone marrow transplantation, and useful as predictive markers for conditions and diseases such as autoimmune disorders, infertility and allergy.

HLA typing is critical for matching donor and recipients for bone marrow transplantation; the use of well-matched donors increases survival and decreases graft vs. host disease. HLA typing is also important for solid organ transplantation, as well as other areas of use.

Unraveling a highly complex HLA type to a molecular level has many challenges. Many molecular typing platforms have been initially tested on HLA genes to determine their potential performance. A successful typing platform involves expertise in probe and primer design in combination with suitable software to interpret the data to achieve correct results.

HLA-G is a special HLA and has been implicated in various immune-mediated diseases and conditions. Specific variants have been reported as associated with risks of asthma, allergy, miscarriage, and preeclampsia. HLA-G is a novel HLA gene that has limited polymorphism in the coding region and a restricted tissue distribution. The HLA-G gene was first identified in 1987 but it was not discovered until 1990 that this gene encoded the unusual HLA molecule that was present in fetal placental cells at the maternal-fetal interface. It was subsequently shown that this one gene encodes at least 7 different protein isoforms, but specific functions of each and the natural history of their expression pattern during pregnancy are still largely unknown. Functionally, HLA-G is thought to play a central role in the maintenance of maternal immunologic tolerance to the genetically foreign fetus. It has been shown to specifically inhibit or activate maternal natural killer (NK) cells and to inhibit maternal cytotoxic T cells. Thus, it appears that a major function of HLA-G in pregnancy is to inhibit maternal immune responses against genetically foreign fetal cells and to promote the protective T-helper (Th) 2 cytokine milieu that characterizes normal pregnancy.

The HLA-G gene has limited variation (polymorphism) in its coding region, particularly compared with the other HLA genes, which are the most polymorphic in the human genome. Only 3 polymorphisms cause amino acid changes in the protein sequence and none have been shown to affect protein function. In contrast, a polymorphic variant (called 1597ΔC) is a frameshift mutation and chromosomes carrying this variant do not make any of the two most abundant isoforms, called G1 and G5. This variant is present and in high frequencies in people of African ancestry and is a null for the G1 and G5 isoforms. The 1597ΔC variant has been shown to be associated with recurrent miscarriage. Because of the relative paucity of variation in the HLA-G gene, few groups worldwide study the genetics of this unusual HLA gene.

HLA-G and Reproduction

Approximately 15% of recognized pregnancies end in miscarriage, making it the most common disorder in pregnancy. Moreover, 2-3% of married couples experience 3 or more miscarriages and often do not have any successful pregnancies. The underlying causes for multiple miscarriages are unexplained in half of these couples. Approximately 10% of married couples are infertile (unable to achieve pregnancy), and in about half of couples it is also unexplained. Preeclampsia is a devastating condition that occurs in 5-8% of all pregnancies and is associated with increased maternal and fetal morbidity and mortality. The causes of preeclampsia also remain unknown. The pathophysiology of all three conditions may be related to defects in implantation in some cases. Recently, HLA-G has been implicated in all three conditions further supporting the notion of a common etiology. For example, reduced levels of HLA-G mRNA and protein have been associated with implantation failure after in vitro fertilization (IVF) and with preeclampsia. Polymorphisms in the HLA-G gene have also been associated with miscarriage and preeclampsia, suggesting that polymorphisms in this gene influence expression levels and pregnancy outcome.

HLA-G and Asthma

Asthma affects nearly 14 million people worldwide and has been steadily increasing in frequency for the past 50 years. Although environmental factors clearly influence the onset, progression, and severity of this disease, family and twin studies indicate that genetic variation also influences susceptibility. Linkage of asthma and related phenotypes to chromosome 6p21 has been reported in seven genome screens. Recently, HLA-G has been identified as an asthma and bronchial hyperresponsiveness (BHR) susceptibility gene on 6p21 and the expression of a soluble isoform (G5) was present in bronchial epithelial cells, expanding this gene's role to include an immune mediated condition.

HLA-G and Adult Cells

The expression of HLA-G protein in adult cells has been implicated in a number of immune-mediated conditions that involve inflammation or tolerance. For example, HLA-G protein was present in bronchial epithelial cells in the asthmatic lung, in muscle fibers from patients with inflammatory myopathies, in T cell infiltrates in skin cells from patients with atopic dermatitis, and in intestinal epithelial cells of patients with ulcerative colitis. Its immunomodulatory role is also reported in patients undergoing transplants, where detection of HLA-G in the circulation and in the transplanted organ is correlated with increased success rates; moreover, tumors that express HLA-G are more likely to escape immune surveillance by the host.

MicroRNAs (miRNAs) are small (˜22 nt) endogenous regulatory molecules that can degrade mRNA or suppress protein translation. There are 326 known miRNAs but there could be 1,000 negatively regulating genes. They negatively regulate gene expression, bind to 3′UTR target sequences in genes and can degrade mRNA or inhibit translation.

MicroRNAs are involved in diversified functions, including development and differentiation, apoptosis, tumorigenesis, insulin secretion, cancer, and viral defense. Human miRNAs can target and bind intracellular viruses, such as HepC and HIV. Some viruses can suppress human miRNA pathways. Some viruses (EBV, herpes) express miRNAs.

A single nucleotide polymorphism or (SNP) is a DNA sequence variation occurring when a single nucleotide in the genome (or any other genetic sequence) differs between members of a species (or between paired chromosomes in an individual). Variation in the nucleotide sequence includes insertion/deletion (“indel”) or substitution. These variations result in two or more alleles depending on the number of polymorphisms in a specific loci. These alleles then form the basis for a genotype of an individual member of the species. Within a population, SNPs are generally assigned an allele frequency, i.e., the ratio of chromosomes in the population carrying a particular variant as compared to the more common variant. Within human populations, a SNP that is more common in one geographical or ethnic group may be much rarer in another.

SNPs may fall within coding sequences of genes, noncoding regions of genes, or in the intergenic regions between genes. SNPs within a coding sequence may not always result in an amino acid change of the protein that is produced, due to redundancy in the genetic code. A SNP which results in the same polypeptide sequence (as compared to the more common variant) is termed synonymous. If a different polypeptide sequence is produced, then the SNPs are referred to as non-synonymous. SNPs that are not in protein coding regions may still have consequences for gene splicing, transcription factor binding, RNA stability or any other transcription and post-transcriptional effects.

### SUMMARY

A SNP (single nucleotide polymorphism) in the 3′ untranslated region (UTR) of HLA-G influences the targeting of three miRNAs to this gene. Allele-specific targeting by miRNA is a novel mechanism for regulating asthma susceptibility genes. MicroRNA (miRNA) target sites in HLA-G were identified and the relationship between miRNA regulation of HLA-G and asthma was elucidated.

HLA-G is a class I HLA molecule that has important immunomodulatory properties. HLA-G was identified as an asthma susceptibility gene and the risk for asthma in the child was found to be determined by both the child's HLA-G genotype and the mother's affection status.

A SNP disrupts a miRNA target site in HLA-G; the SNP in the miRNA target site is associated with asthma and respiratory viral illness in childhood. Infection of bronchial epithelial cells with rhinovirus down regulates expression of miRNAs that target HLA-G in some individuals.

Analysis of miRNA interference with HLA-G expression identified the HLA-G 3′UTR SNP+3142G/C that disrupts a target for miRNA 148 and is associated with asthma. The polymorphism is associated with protection from (or susceptibility to) moderate to severe viral infection in the first 3 years of life, with an interaction with mother's affection status (asthma).

HLA-G 3′UTR is a target site for miR-148a, -148b, and -152. The +3142 C allele disrupts targeting, while the +3142 G allele retains targeting. HLA-G+3142 C/G may be associated not only with asthma but with associated phenotypes. Interaction between maternal asthma and offspring's HLA-G genotypes influences response to viral infections and asthma risk in the children.

A method for controlling or suppressing asthma or viral infection includes the steps of:

(a) providing a nucleotide molecule or a microRNA molecule capable of interacting with HLA-G; and

(b) controlling or suppressing asthma or viral infection by regulating HLA-G expression with the microRNA molecule.

A method of identifying a risk factor for asthma includes the steps of:

(a) determining whether a HLA-G allele includes a cytosine or a guanine at position +3142 of HLA-G gene; and

(b) establishing that the cytosine at position +3142 is a risk factor for asthma and protective against viral illness in children of mothers with asthma, and that the guanine at position +3142 is a risk factor for asthma and protective against viral illness in children of mothers without asthma.

HLA-G alleles that predispose to asthma also protect against illness with RV infection in childhood, and mother's affection status determines which allele is associated with risk. In offspring of mothers with asthma the risk allele is +3142C whereas in offspring of mothers without asthma the associated allele is +3142G. The relative expression of +3142G to +3142C mRNA or protein is influenced by the presence of miR148.

### BRIEF DESCRIPTION OF THE DRAWINGS

FIG. 1 shows luciferase reporter assays in HBE cells and HPEG2 cells.

FIG. 2 shows the expression of miR-148a via northern blot and real-time PCR in various cell types.

FIG. 3 shows the specific variation in HLA-G that is associated with susceptibility to asthma. The +3142 variation is shown within a putative microRNA target site.

FIG. 4 shows that HLA-G+3142 C/G resides within a targeting site of miR-148a, 148b, 152, and that the C allele disrupts the site.

FIG. 5 is a schematic illustration of luciferase constructs for testing miRNA targeting.

FIG. 6 shows the results of luciferase assays in 16HBE cells for the +3142 G/C variation.

FIG. 7 shows proportions of maternal asthma, +3142 C/G genotype, and respiratory viral illness in COAST children.

FIG. 8 shows the interaction between HLA-G genotype, maternal asthma, and respiratory viral illness in year 2.

FIG. 9 HLA-G+3142C/G affects the targeting of miR-148a, miR-148b and miR-152 to HLA-G and interacts with mother's asthma status to determine risk for asthma in the child. (a) Predicted binding of miR-148a to HLA-G. The seed region of the target site is shown in bold letters, +3142C/G is in a box. The RNA duplex was analyzed by RNA hybridization. (b) Luciferase assays showing the allele-specific targeting of the 3 miRNAs to HLA-G in 16HBE14o-cells. RLU=Relative Light Unit. +3142G or +3142C luciferase plasmid was cotransfected with negative control miRNA, or miR-148a, miR-148b and miR-152. The P-values for the difference between the 2 plasmids are shown under each panel. (c) Endogenous HLA-G can be inhibited by miR-148a in JEG3 cells. JEG3 cells, which are +3142GG, were transfected with miR-148a, with negative control miRNA, or with HLA-G siRNA as a positive control. miR-148a and HLA-G siRNA significantly reduced the level of soluble (s)HLA-G in JEG3 supernatant compared to the NC miRNA. (d) +3142C/G interacts with maternal asthma status and is associated with asthma in the COAST children. Dashed lines are the children of mothers with asthma (N=58) and solid lines are the children of mothers without asthma (N=119). The interaction P-value=0.0011.

FIG. 10 shows pairwise linkage disequilibrium (r²) map of HLA-G in the Chicago families. The LD plot was made by Ldplotter (http://innateimmunity.net/IIPGA2/Bioinformatics/).

FIG. 11 illustrates predicted binding of miR-148b (a) and miR-152 (b) to HLA-G. The seed region of the target site is shown in bold letters, +3142C/G is highlighted in yellow. The MFE of the RNA duplex was analyzed by RNAhybrid.

FIG. 12 shows miR-148a (a) and miR-148b (b) levels in primary cytotrophoblast cells (CTB) from six individuals and primary bronchial epithelial cells (BE) from three individuals determined by miRNA real-time PCR. miR-148a (P=0.0016) and miR-148b (P=0.0006) levels were significantly lower in CTB cells than in BE cells.

### DETAILED DESCRIPTION

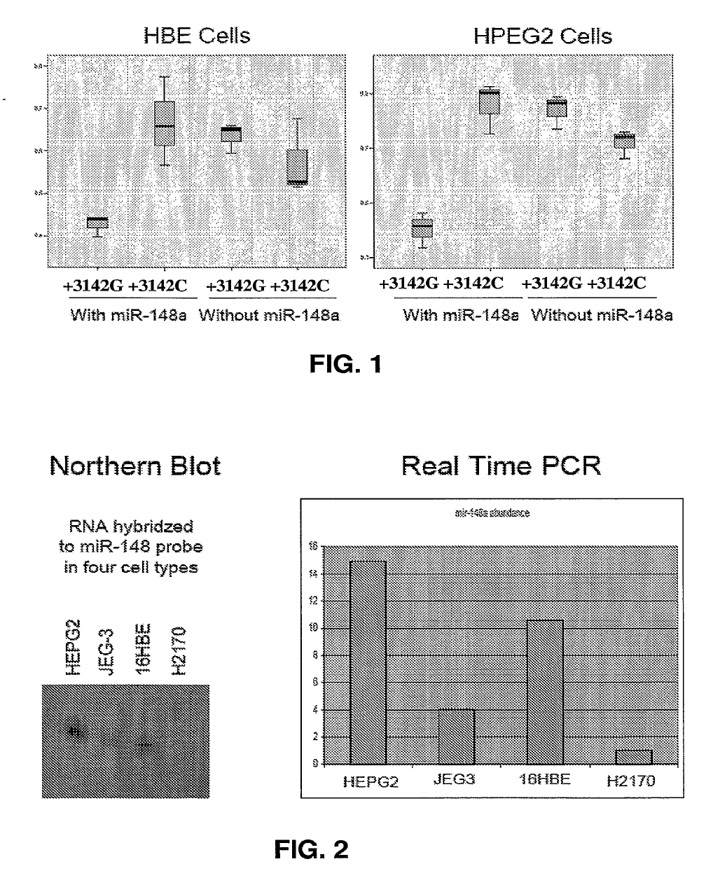

MicroRNAs (miRNAs) are small (˜22 nt) RNA molecules that negatively regulate genes at the post-transcriptional level by degrading miRNA or suppressing protein translation. Potential miRNA target sites in HLA-G, identified as an asthma gene were searched), and the relationship between HLA-G polymorphisms, miRNA regulation, and asthma were analyzed. The HLA-G gene sequence was scanned for miRNA target sites based on sequence matching and analysis of RNA secondary structures, and a target site for miR-148a, -148b and -152 was found in the HLA-G 3′-UTR that contained a SNP (+3142 C/G) in the seed region. The 3′UTR of HLA-G was cloned into a luciferase vector, and mutated so that one had a +3142C and one a +3142G. The vectors were transfected into human bronchial epithelial cells (HBE) with and without the miRNA mir-148 (FIG. 1). If the sequence with the +3142G is a target site for mir-148, expression should be reduced in cells transfected with the G all and mir-148, as demonstrated in this experiment.

Luciferase assays in a human bronchial epithelial (HBE) and liver (HPEG2) cell lines showed that the C allele, which disrupted this putative target site, had 1.3-1.5 times more luciferase activity than the G allele (p<0.05) when co-transfected with one of the three miRNAs, thus validating the predicted target site (FIG. 1). In contrast, the C allele showed significantly lower expression than the G allele in primary BE cells from two heterozygous individuals. The +3142C/G SNP was associated with asthma in several populations (p<0.05). Genotype-specific regulation of HLA-G expression by miRNA might be involved in the pathogenesis of asthma.

A mir-148 target site was predicted in the 3′-UTR of HLA-G that contained a SNP (+3142 C/G). The C allele, which disrupted the putative target site, was associated with asthma in the African Americans (AAs) (P=0.02) and had significantly increased luciferase activity over the G allele (P<0.01) in a human bronchial epithelial cell line. The presence of mir-148 in these cells was confirmed by northern blot analysis (FIG. 2).

The HLA-G sequence was scanned for miRNA target sites by bioinformatics methods based on sequence matching to miRNA and RNA secondary structure analysis. Genotyping was performed using a Taqman assay in African Americans (AAs) asthma cases (N=159) and controls (N=160) and in European Americans (EAs) asthma cases (N=99) and controls (N=180). The HLA-G 3′-UTR was cloned into a luciferase vector, and site-directed mutagenesis was performed to mutate the predicted miRNA target site. Luciferase activities for the two constructs were analyzed in four different cell lines.

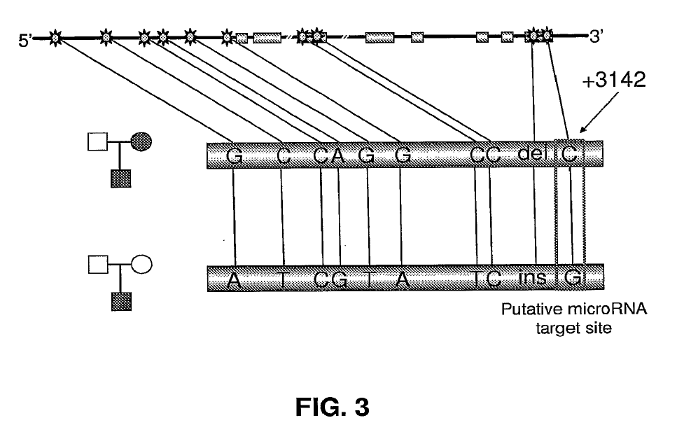

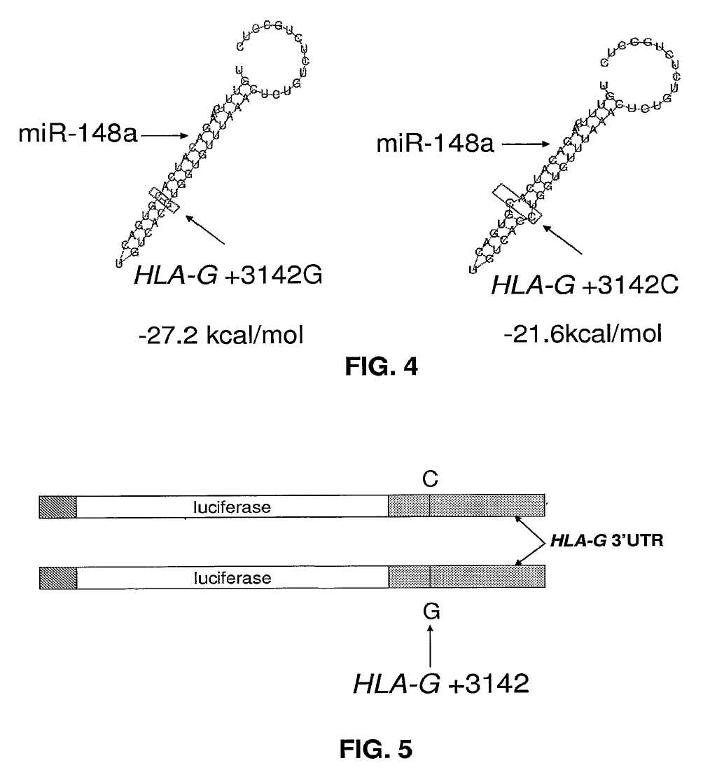

A novel variation in HLA-G and susceptibility to asthma, the +3142 variation, is shown within a putative microRNA target site (FIG. 3). The HLA-G+3142 C/G influences targeting of miR-148a, 148b, 152. In the 3′-UTR of HLA-G, a target site for 3 miRNAs, miR148a, 148b, and 152 was identified (FIG. 4). The +3142 C/G is located in the seed region or core region of the target site, which is identical for all 3 miRNAs. The structure of miR-148a (the left strand) and its HLA-G target (the right strand) is shown (FIG. 4). When there is a G allele at +3142, the seed region of the target site is complementary to 148a, and this binding is stable based on the calculation of the free energy. When there is a C allele at +3142, there is a mismatch between the seed region of the target site and 148a, and therefore the binding is not stable.

A schematic illustration of luciferase constructs for testing miRNA targeting is shown in FIG. 5. About 260 bp of the HLA-G 3′-UTR (almost the entire 3′-UTR) are inserted into a luciferase vector, and site-directed mutagenesis is used to create 2 plasmids, the C plasmid and the G plasmid, that differed only at +3142. These vectors were then transfected into a human bronchial epithelial cell line, 16HBE.

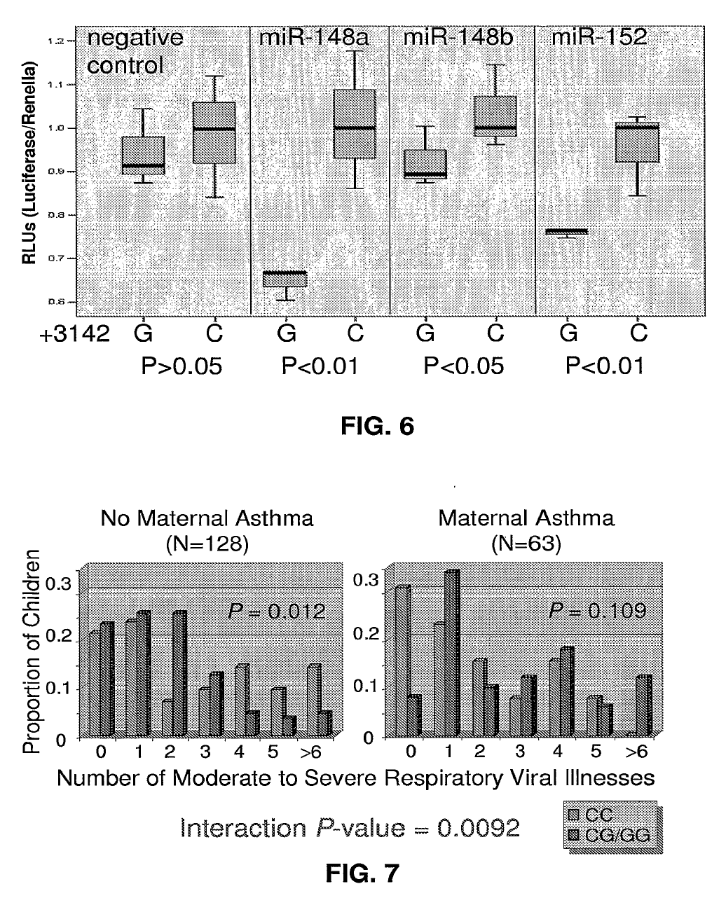

The results of luciferase assays in 16HBE cells for the +3142 G/C variation are shown in FIG. 6. The G or C plasmid was co-transfected into 16HBE cells, with either the negative control miRNA, or microRNA 148a, 148b or 152. The negative control miRNA is a commercially available small RNA that does not target any human gene. Each plot represents the results of 6-9 replicate experiments. The expression of the G and C plasmids were similar when co-transfected with the negative control miRNA. But when co-transfected with either one of the 3 miRNAs that target HLA-G, the expression of the G plasmid was significantly lower than the expression of the C plasmid (FIG. 6).

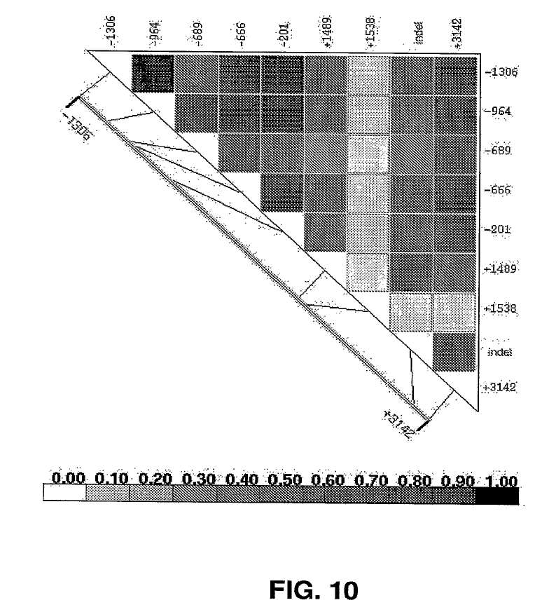

The relationship between maternal asthma, the child's +3142 C/G genotype, and respiratory viral illness in COAST children are shown in FIG. 7. Data shows the association of +3142 C/G genotype with the number of moderate to severe viral respiratory illnesses in the second year of life, stratified by the mothers' asthma status. In 128 children of mothers without a history of asthma. the CC individuals (light) had significantly more viral infections than children carrying a G allele (dark) (FIG. 7). In contrast, the pattern was the opposite among 63 children of mothers with asthma, the CC children have less viral infections than the children with a G allele. Although these differences were not significant in this small sample, there was a significant interaction between mother's asthma status, child's genotype, and number of viral illnesses, similar to earlier studies in Chicago and Dutch families (FIG. 10).

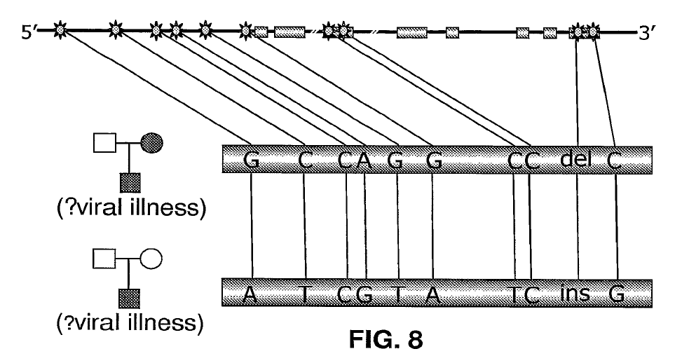

HLA-G, maternal asthma, and respiratory viral illness in year 2 are shown in FIG. 8. Because respiratory viral illness with wheezing is a risk factor for the development of asthma, it was predicted that among children of asthmatic mothers, the C allele would have been associated with more viral illnesses. But the opposite was observed, in that, these children had fewer viral illnesses, while there were fewer viral infections among G children of unaffected mothers. Children with fewer viral illness in childhood may be at higher risk for asthma.

HLA-G was identified as an asthma gene in a positional cloning study in Chicago Caucasian families with at least 2 children with asthma. In both the Chicago families and in a Dutch population, the risk genotype at HLA-G varied depending on whether the mother had asthma or bronchial hyperresponsiveness (BHR) (affected) or was unaffected. For example, in both populations, the GG genotype at a SNP in the promoter region of HLA-G, −964G/A, was associated with asthma in children with affected mothers; whereas the AA genotype was associated with asthma in children with unaffected mothers. A similar interaction was observed with +1489C/T, the SNP that explained nearly the entire linkage signal in the Chicago families. In the highly polymorphic promoter region, the −964G/A SNP tagged two major haplotype clades that have under long standing balancing selection. Functional differences were not found between these 2 promoter clades using luciferase reporter assays. Expression of soluble (s)HLA-G protein in bronchial epithelial cells in 2 individuals with asthma but not in 1 normal control individual, suggested that this gene was upregulated in the asthmatic lung.

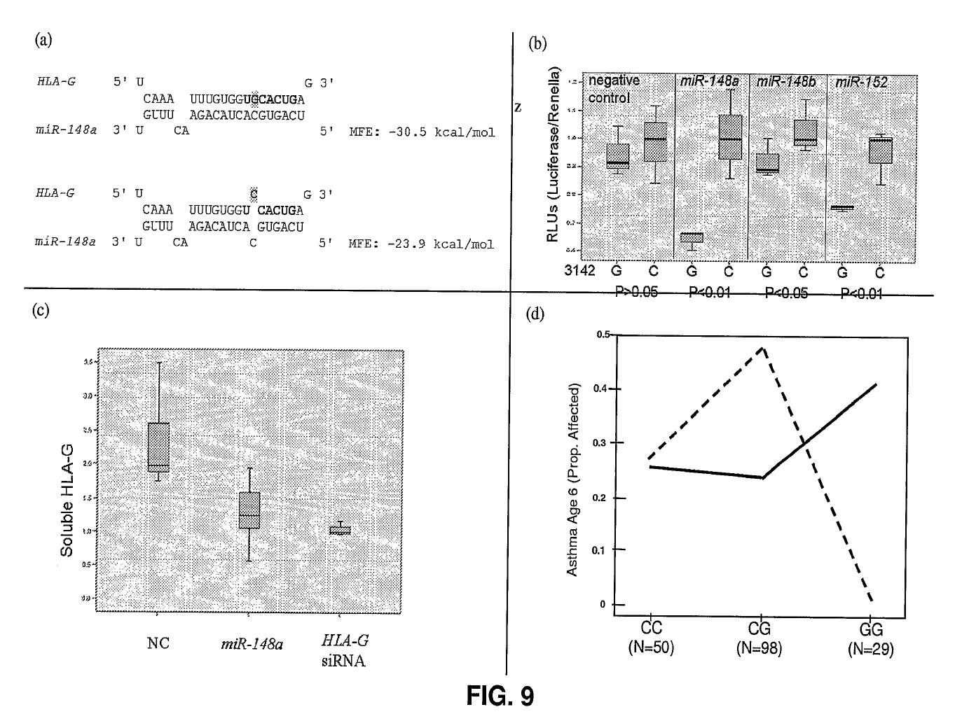

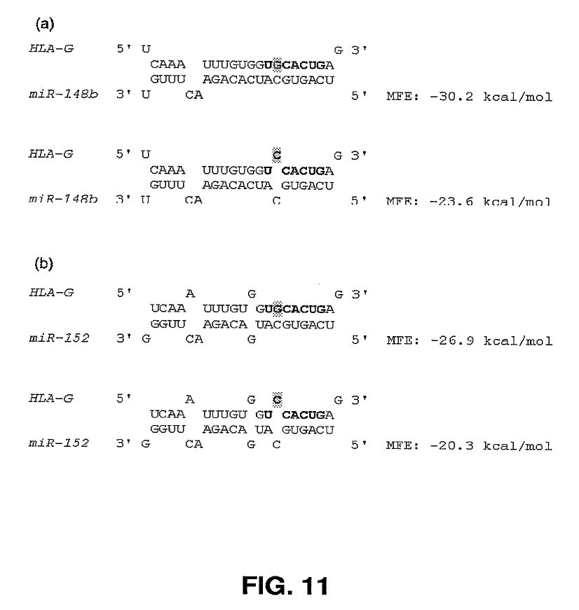

There is strong linkage disequilibrium (LD) among SNPs in the HLA-G gene (FIG. 10). Other regions of the gene were examined to identify variation that contributes to asthma risk. A putative target site for 3 miRNAs was identified in the 3′-UTR of HLA-G: miR-148a, miR-148b and miR-152. A C/G SNP at +3142 in the HLA-G message (dbSNP accession number rs1063320) is located in the seed region of this target site. Based on the calculation of minimum free energy (MFE), the binding of miRNA to the G allele is predicted to be more stable than binding to the C allele (FIG. 9a; FIG. 11). Consequently, +3142 could influence the binding of miRNA to HLA-G, and therefore HLA-G protein expression, in an allele-specific manner.

In order to test allele-specific effects on miRNA targeting, the HLA-G 3′-UTR was cloned into a luciferase vector, and a “G” plasmid and a “C” plasmid were created that differed only at +3142C/G. Luciferase assays were performed by cotransfecting the “G” or “C” plasmid with either miR-148a, miR-148b, miR-152, or a negative control miRNA (NC miRNA) into a human bronchial epithelial cell line, 16HBE14o-. The normalized luciferase readings of the “G” plasmid were significantly lower than those of the “C” plasmid when cotransfected with each of the 3 miRNAs, but the luciferase readings of the 2 plasmids were not significantly different when cotransfected with the NC miRNA, indicating allele-specific targeting of the 3 miRNAs to HLA-G (FIG. 9b).

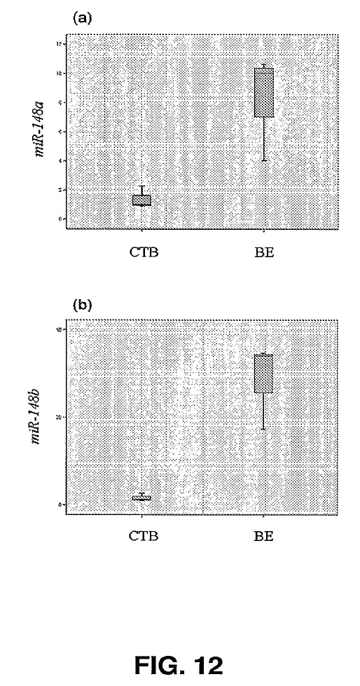

To further validate the miRNA target site in its native form, a choriocarcinoma cell line, JEG3, was used. JEG3 cells constitutively expresses very high levels of HLA-G and have the genotype GG at +3142. JEG3 cells were transfected with miR-148a, the NC miRNA (negative control), or HLA-G siRNA (positive control), and sHLA-G was measured in the supernatant by ELISA. sHLA-G levels were significantly decreased by miR-148a and by HLA-G siRNA, compared to the NC miRNA transfection (P=0.019 and 0.0048, respectively), confirming the presence of a functional target site on the +3142G allele (FIG. 9c). miR-148a and miR-148b but not miR-152 were detected in primary bronchial epithelial cells and to a less extent in primary cytotrophoblast cells by real time PCR (FIG. 12). The higher expression of these miRNAs in bronchial epithelial cells than in cytotrophoblast cells is consistent with the expression of HLA-G, which is low in the former and high in the latter cells.

HLA-G+3142C/G is in high LD with the HLA-G promoter polymorphism that was associated with asthma in Chicago and Dutch families (FIG. 10). Therefore, it is likely that +3142C/G accounts for at least some of the previously observed association. To further explore this possibility, a third population was studied consisting of children participating in a birth cohort study. Couples in which at least 1 parent had asthma or allergies were recruited into the Childhood Origins of ASThma (COAST) study in Madison, Wis. during pregnancy. Their children are at high risk for developing asthma and have been followed from birth through age 6, at which time asthma is diagnosed. 180 Caucasian COAST children were genotyped for 4 polymorphisms in HLA-G: −1306G/A, which is in perfect LD with the promoter SNP (−964G/A) that was associated with asthma in the Chicago and Dutch families (FIG. 10); +1489C/T, a nonsynonymous SNP in exon 3 that explained most of the evidence for linkage in the Chicago families; a 14 base pair insertion/deletion (indel) in the 3′UTR that has been associated with mRNA stability, and +3142C/G. Significant interactions (P<0.01) were observed at all SNPs except +1489C/T (Table 1), between mother's affection status (asthma vs. no asthma) and the child's genotype on the development of asthma by age 6 in this cohort. However the association with +3142C/G was the most significant (permutation P_{interaction}=0.0011). In the COAST children, the GG genotype is protective against asthma among children of asthmatic mothers (0 of 20 children; P=0.0036) but is associated with modest risk for asthma among children of nonasthmatic mothers (7 of 32 children; P=0.39) (FIG. 9d, Table 1). Therefore, the +3142G allele, or promoter alleles in strong LD with +3142G (r²=0.76 between +3142 and either −964 or −1306), is a susceptibility allele among children of asthmatic mothers, and the C allele may be a risk allele (albeit more modest) among children of nonasthmatic mothers.

Gene-environment interactions play an important role in the etiology of common diseases. Genes with main effects on disease risk may be more the exception that the rule. Identification and characterization of such interactions is critical for both basic research and clinical practice by helping to explain some of the inconsistencies of disease association studies and by identifying individuals with genotype specific risks for detrimental environmental exposures. A functional polymorphism in HLA-G interacts with mother's asthma status, which represents an “in utero” environmental exposure. This interaction has now been observed in 3 independent Caucasian populations ascertained using 3 different study designs: families ascertained through affected sib pairs in the Chicago study, families ascertained through an affected parent originally studied 20 years earlier in the Dutch study, and children participating in a birth cohort study. Thus, the finding of an interaction between maternal affection status and child's HLA-G genotype seems robust to study design and is particularly intriguing for two reasons. First, maternal asthma remains the most significant and best replicated risk factor for asthma in her children, and second, HLA-G is most highly expressed during pregnancy where it is thought to play a key role in modulating immune tolerance toward the genetically foreign fetus by enhancing the Th2 arm of the immune system. Asthma and allergic disease are also characterized by a skewing toward Th2 immunity. As disclosed herein, maternal asthma influences child's risk in an allele-specific manner and that the immunosuppressive (Th2-skewing) properties of HLA-G promotes asthma pathogenesis.

Fetal miRNA regulations may differ in pregnancies of asthmatic and non-asthmatic mothers. The fetal lung is bathed in amniotic fluid that is swallowed by the fetus, and could provide a direct route for maternal factors to modulate fetal gene expression in the lung as an epigenetic phenomenon.

The discovery that 3′-UTR polymorphisms affects miRNA targeting reveals a novel functional mechanism for non-coding polymorphisms. The number of human miRNAs could be as high as 25,000 and could regulate up to 90% of human genes. The involvement of miRNA regulation in human diseases may be quite prevalent and particularly relevant to common diseases, for which many susceptibility loci with small-effects on gene expression likely contribute. Allele-specific miRNA targeting may be a common mechanism of human disease pathogenesis.

### EXAMPLES

### Example 1

Luciferase assays in HBE cells. Luciferase expression in +3142C cells contransfected with miR148a is greater than in +3142G cells cotransfected with miR148a. Luciferase expression in +3142C without miR148a similar to +3142G without miR148a.

### Example 2

Northern and qRT-PCR of miR148 in BE cells. miR148 is expressed in HBE and other cell lines of epithelial origin (FIG. 2). This is the first demonstration that a common SNP disrupts a miRNA target site in humans.

### Example 3

Association studies. +3142C is associated with asthma in Chicago (African Americans (P=0.02). +3142C is associated with asthma in Chicago and Dutch children of mothers with asthma but +3142G is associated with asthma in Chicago and Dutch children of mothers without asthma. (FIG. 10) +3142C is protective against RV illness in COAST children of mothers with asthma but +3142G is protective against RV illness in COAST children of mothers without asthma (FIG. 7).

This is the first demonstration that a SNP in a miRNA target site is associated with asthma (or any common disease) and the first demonstration that a SNP in a miRNA target site is associated with respiratory viral illness in childhood. Some data suggests that infection of bronchial epithelial cells with rhinovirus down regulates expression of miRNAs that target HLA-G, a novel observation.

### Example 4

In a cohort study of children at high risk for developing asthma, a significant association was found between this the SNP in the miRNA target site and number of moderate to severe respiratory viral illnesses in childhood (P=0.0141).

Children with at least 1 parent with allergy or asthma recruited prior to birth were studied. Extensive phenotyping in years 1-3 were performed. Assessment of viral illness, cytokine response, wheezing illness were carried out. The +3142 C/G genotypes were analyzed in children in the COAST study. (COAST, Childhood Onset of ASThma, is a prospective cohort study conducted in Madison by Dr. Rob Lemanske.) The COAST children are at high risk for developing asthma, at least one of their parents have allergy or asthma. And the COAST children have been extensively phenotyped from birth through year 3. Because of previous findings in the Chicago and Dutch families, these data were analyzed, stratified by maternal asthma status, and examined for associations between +3142 C/G and asthma-related outcomes in the first few years of life.

TABLE 1
Interaction of HLA-G genotypes and maternal asthma status on asthma risk in COAST
children. The minor allele frequency at each polymorphism in the COAST children is
0.395 (−1306A), 0.293 (+1489T), 0.372 (exon 8 ins), and 0.461 (+3142G).

| | | Mothers' Affection Status | | | | |
|---|---|---|---|---|---|---|
| | | Asthma+ | | Asthma− | | |
| | | Children's Affection Status | | | | P Value of |
| Number of Children with Genotype of | | Asthma+ | Asthma− | Asthma+ | Asthma− | Permutation test |
| HLA-G−1306 | GG | 6 | 10 | 14 | 35 | 0.0045 |
| | AG | 14 | 15 | 11 | 43 | |
| | AA | 0 | 11 | 5 | 9 | |
| HLA-G+1489 | CC | 9 | 16 | 18 | 44 | 0.0978 |
| | CT | 10 | 12 | 9 | 28 | |
| | TT | 0 | 3 | 4 | 4 | |
| HLA-G exon8 indel | DelDel | 6 | 15 | 14 | 38 | 0.0142 |
| | InsDel | 14 | 14 | 14 | 44 | |
| | InsIns | 0 | 9 | 4 | 8 | |
| HLA-G+3142 | CC | 3 | 8 | 10 | 29 | 0.0011 |
| | CG | 17 | 18 | 15 | 48 | |
| | GG | 0 | 12 | 7 | 10 | |

Materials and Methods

Software analysis: The HLA-G sequence was scanned for miRNA target sites by bioinformatics methods based on sequence matching to miRNA and RNA secondary structure analysis. Genotyping was performed using a Taqman assay in African Americans (AAs) asthma cases (N=159) and controls (N=160) and in European Americans (EAs) asthma cases (N=99) and controls (N=180), as well as in a birth cohort of Caucasian children (N=180). The HLA-G 3′-UTR was cloned into a luciferase vector, and site-directed mutagenesis was performed to mutate the predicted miRNA target site. Luciferase activities for the two constructs were analyzed in four different cell lines.

Every possible miRNA target site in HLA-G was checked by analyzing: (i) optimal and sub-optimal base pairing between miRNA and target mRNA of the binding site and (ii) optimal and sub-optimal energies and secondary structures in pairing and loop regions.

Based on the results, the target site for miR-148a, 148b and 152a was predicted with 90% confidence by comparing the wild type and mutant alleles at +3142 C/G by structure and energy analysis. This target site could be disrupted by single nucleotide change from G to C.

Details about the prediction of microRNA targeting HLA-G:

Step 1: The target of microRNA is predicted mainly based on the score of:

1. Minimal requirement of 5′ end of microRNA:target mRNA pairing.

2. Over all microRNA: target mRNA binding energies cut off.

3. mRNA secondary structure in microRNA target regions.

Step 2: After running the program with all microRNAs sequences (more than 300 microRNA entries), every possible microRNA target site was checked one by one by analyzing.

1. Optimal and suboptimal base pairing between microRNA and target mRNA at binding site.

2. Optimal and suboptimal energies and secondary structures in pairing and loop regions.

3. Relative positions of microRNA target sites in 3′UTR.

The prediction of which microRNA regulated HLA-G used all of the criteria described above. The program is used is just a tool to make the prediction work more efficiently.

Polymorphism (+3142 C/G) was identified in the target site of miR-148 in dbSNP. By comparing the wild type and mutant (SNP, C/G) through structure and energy analysis, it was predicted that the regulation of HLA-G by miR-148 family should be abolished by single nucleotide change from G to C at +3142 with a 90% confidence.

Wild type and mutant HLA-G 3′UTR fragments were cloned into luciferase reporters and transfected into different cell lines. A few cell lines related to asthma were screened by Northern blot and found that human bronchial epithelial (BE) cells naturally express miR-148 at high abundance. It was also found the C allele had 1.6-fold more luciferase activity than the G allele (P<0.01) in BE cells.

These results validated the prediction and the hypothesis that the +3142 C allele is associated with higher expression of HLA-G. Genotype-specific regulation of HLA-G expression might be involved in the pathogenesis of asthma.

Two published miRNA prediction programs (Kiriakidou et al., 2004; Lewis, 2003) did not identify any miRNA binding regions.

The software analysis may be performed using any software module. The term “module” or “computer module” or “software module” referenced in this disclosure is meant to be broadly interpreted and cover various types of software code including but not limited to routines, functions, objects, libraries, classes, members, packages, procedures, methods, or lines of code together performing similar functionality to these types of coding. The components of the present disclosure are described herein in terms of functional block components, flow charts and/or various processing steps. As such, it should be appreciated that such functional blocks may be realized by any number of hardware and/or software components configured to perform the specified functions. For example, the present disclosure may employ various integrated circuit components, e.g., memory elements, processing elements, logic elements, look-up tables, and the like, which may carry out a variety of functions under the control of one or more microprocessors or other control devices. Similarly, the software elements of the present invention may be implemented with any programming or scripting language such as C, C#, SQL, C++, Java, COBOL, assembler, PERL, or the like, with the various algorithms being implemented with any combination of data structures, objects, processes, routines or other programming elements. Further, it should be noted that the present disclosure may employ any number of conventional techniques for data transmission, signaling, data processing, network control, and the like as well as those yet to be conceived. The module may be stored on any known computer readable medium or delivered over any known data transmission signal and like known or yet to be conceived as well.

Construction of Luciferase Reporter Plasmids for the Verification of miRNA Target Site:

1. Amplification of HLA-G 3′-UTR region: Forward primer: ctagaagctttgtgaaacagctgccc, reverse primer: cgctagtctagatgtctctcaaatftc. 50 μl PCR reactions were performed with 10 ng template DNA, 5 μl 10×PCR minus Mg buffer, 4 μl 2.5 mM dNTP mix, 3 μl forward primer (10 μM), 3 μl reverse primer (10 μM), 3 μl 25 mM MgCl₂, 0.3 μl Platinum Taq DNA polymerase (5 U/μl, Invitrogen).

PCR cycling conditions: 95° C. 10′, followed by 10 cycles of 95° C. 10″, 54° C. 30″, and 72° C. 30″; then another 25 cycles of 95° C. 10″, 61° C. 30″, and 72° C. 30″, followed by 72° C. for 7′ and hold at 4° C. The DNA template used has the insertion allele of the HLA-G exon8 14 bp indel, and has G at +3412.

2. The PCR product was digested by HindIII and XbaI (the HindIII and XbaI restriction sites are at the forward and reverse primer, respectively), and cloned to pLS-HX (provided by Ligang Wu, New York University).

3. Mutant plasmid was constructed by QuikChange XL site-directed mutagenesis kit (Stratagene). Mutation primers: forward: ctctgtctcaaatttgtggtccactgagctataacftacttc, reverse: gaagtaagttatagctcagtggaccacaaatttgagacagag.

Luciferase assay: 16 HBE cells were seeded to 12-well plate the day before transfection and grown to 30-40% confluence. For each transfection, 25 ng luciferase plasmid, 500 pg renella plasmid (provided by Ligang Wu, New York University) and 3 ul of lipofectamine 2000 (Invitrogen) were used. MicroRNA (Ambion) was added to a final concentration of 10 nM. Dual-luciferase reporter assay kit (Promega) was used to measure the luciferase and renella readings.

Real-time PCR: mirVana qRT-PCR miRNA Detection Kit (Ambion), together with mirVana qRT-PCR primers sets for hsa-miR-148a, hsa-miR-148b, hsa-miR-152, and human U6, were used for the quantification of miRNA levels by real-time PCR.

MiRNA northern blotting: 10-20 ug RNA of each sample was denatured 1/1 in Gel loading buffer II (Ambion 95% formamide, 0.025% xylene cyanol, 0.025% bromophenol blue, 18 mM EDTA, and 0.025% SDS) at 95-100° C. for 5 min and separated by 15% 8M Urea PAGE (17×10 cm gel, Acr:Bis 19:1) until bromophenol blue dye run near gel bottom. tRNA on the upper part of PAGE gel can be cut and stained with EB as loading control.

RNA samples were electrophoretically transferred (3 mA/cm2, 0.5A for 15 min, then 20V for 25 min) to Hybond-XL nylon membrane (Amersham). Then the membrane was crosslinked by UV 2400uj/cm2.

DNA oligo complimentary to miRNA was labeled by T4 polynucleotide kinase with γ-P³²-ATP. The reaction was set up as follows:

| | |
|---|---|
| H2O | 10 |
| DNA oligo (10 pmol/ul) | 1 |
| PNK 10Xbuffer | 2 |
| [γ-P³²]ATP (3000 Ci/mmol) | 5 |
| T4 polynucleoacid kinase (10 U/ul) | 2 |
| | 20 ul |

The reaction was performed at 37° C. for 60 min. Then the probes were purified by NucAway G25 Spin Columns (Ambion). The probes were denatured at 100° C. for 5 min before use.

Membranes were pre-hybridized at 37° C. for 30 min in 8 ml PerfectHyb Plus hybridization buffer (Sigma). Then radio-labeled DNA probe was add to the hybridization buffer to >=1×10⁶ cpm/ml and hybridized at 37° C. overnight. After hybridization, the blot was washed 2×SSC, 0.5% SDS, once 5 min RT, twice 20 min 30° C. and exposed for 1-2 days.

Identification of miRNA Target in HLA-G

miRNA target in HLA-G was predicted by the algorithm disclosed herein, which is based on the criteria of Watson-Crick base-pair of the “2-8” nucleotides of miRNA to target sequence, and stable binding of miRNA to the target region but not to the flanking region.

Cell Lines and Samples

Human bronchial epithelial cell line 16HBE14o-was provided by Dr. Julian Solway of the University of Chicago and Dr. Steven White of the University of Chicago, human choriocarcinoma cell line JEG3 was purchased from the ATCC. Primary bronchial epithelial cells were collected from surgical specimens of normal human bronchi or trachea (lung transplant donors) at the University of Wisconsin, Madison, the experimental protocol was approved by the University of Wisconsin Human Subjects Committee. Primary trophoblast cells were collected from first trimester placentas obtained at the University of Chicago. Investigations were approved by the Institutional Review Board of the University of Chicago.

Luciferase Assay

A 365 bp region of HLA-G 3′-UTR containing the insertion allele at the indel was amplified by primers CTAGAAGCTTTGTGAAACAGCTGCCC and CGCTAGTCTAGATGTCTCTCAAATTTC, digested by HindIII and XbaI, and ligated into luciferase plasmid pLS-HX. Site-directed mutagenesis was performed using QuikChange II XL kit (Stratagene). 16HBE14o-cells were seeded at 80% confluency in 12-well plate prior to transfection, for each well 50 ng luciferase plasmid and 2 ng renella plasmid were transfected together with either negative control miRNA, miR-148a, miR-148b or miR-152 (Dharmacon) at a final concentration of 20 nM, using Lipofectamine 2000 (Invitrogen) according to manufacturer's protocol. Cells were lysed 40 hours after transfection, and luciferase and renella readings were obtained by using Dual-Luciferase Reporter kit (Promega) and Veritas Microplate Luminometer (Turner Biosystems). At least 6 replicate assays were performed for each transfection. Luciferase readings were normalized by renella readings, and the values were further normalized by the median of the “C” plasmids within each miRNA transfection group.

Inhibiting HLA-G by miR-148a in JEG3 Cells

JEG3 cells were seeded at 80% confluency in 12-well plate prior to transfection, negative control miRNA, miR-148a, or HLA-G siRNA (Dharmacon) was transfected into JEG3 cells at a final concentration of 20 nM using Lipofectamine 2000 according to manufacturer's protocol. Cell supernatants were tested 5 days after transfection using HLA-G ELISA kit (Biovendor & Exbio). A total of 7 replicate assays were performed for each transfection. sHLA-G level was normalized to the median of the HLA-G siRNA group.

miRNA Real Time PCR

miRNA was extracted by mirVana miRNA isolation kit (Ambion) from primary bronchial epithelial cells from 6 individuals and from primary cytotrophoblast cells from 6 placentas. miRNA real time PCRs for miR-148a, miR-148b and miR-152 were performed with 20 ng miRNA and mirVana miRNA detection kit and primer sets (Ambion). As an estimation of the levels of the 3 miRNAs, U6 real-time PCR was also performed for these samples. miR-148a and miR-148b, but not miR-152, were detected in these samples, with the average difference of Ct values being:

bronchial epithelial cells: 6 (miR-148a-U6), 6.4 (miR-148b-U6); trophoblast cells: 9.3 (miR-148a-U6), 11.2 (miR-148b-U6).

Genotyping

HLA-G-1306G/A, +1489C/T, and the exon8 indel were genotyped. HLA-G +3142C/G was genotyped using Taqman Assay-On-Demand (Applied Biosystems). +3142 genotype of JEG3 was determined by amplifying HLA-G 3′-UTR with the same primers used in the luciferase assay and sequencing.

Statistical Analysis

The statistical significance of differences in the luciferase assays, sHLA-G levels in JEG3 supernatants, and miRNA levels between cytotrophoblast cells and bronchial epithelial cells was tested using two-tailed Student's t-test. Fisher's exact test was used to test the association of +3142C/G with asthma in COAST children of asthmatic and non-asthmatic mothers.

The interaction between child's genotype and mother's asthma status in determining child's asthma risk was modeled using logistic regression. To test the significance of this interaction, children's asthma status was permutated 10,000 times, while mothers' status and children's genotypes were unchanged. For each generated dataset, the log likelihood ratio test was performed to compare the model with interaction to the one without interaction. Permutated P value (P_{interaction}) was determined by the number of datasets that had smaller log likelihood P values than the original dataset.

Sequences:

The flanking sequence for SNP+3142 C/G in the HLA-G 3′-UTR is tccgtctctgtctcaaatttgtggtC/Gcactgagctataacttacttctgta.

The miRNA sequences are:

1. hsa-miR-148a; Accession number: MIMAT0000243 (accession number corresponds to the miRBase, http://microrna.sanger.ac.uk). The microRNA sequence for miR-148a is UCAGUGCACUACAGAACUUUGU

2. hsa-miR-148b; Accession number: MIMAT0000759 (accession number corresponds to the miRBase, http://microma.sanger.ac.uk). The microRNA sequence for miR-148b is UCAGUGCAUCACAGAACUUUGU

3. hsa-miR-152; Accession number: MIMAT0000438 (accession number corresponds to the miRBase, http://microrna.sanger.ac.uk). The microRNA sequence for miR-152 is UCAGUGCAUGACAGAACUUGGG

### DOCUMENTS CITED

Kiriakidou M et al. (2004) A combined computational-experimental approach predicts human microRNA targets Genes Dev 18:1165-78;
Lemanske, R. F., Jr. The childhood origins of asthma (COAST) study. Pediatr Allergy Immunol 13 Suppl 15, 38-43 (2002).
Lewis B et al. (2003) Prediction of mammalian microRNA targets. Cell 115:787-98
Martinez, F. D. Maternal risk factors in asthma. Ciba Found Symp 206, 233-9; discussion 239-43 (1997).

1. The use of a nucleotide molecule or a microRNA molecule capable of interacting with HLA-G for the preparation of a medicament for the treatment of asthma or viral infection.

2. Use of HLA-G 3′UTR polymorphism at position +3142 as a predictor of risk factor for asthma.

3. A composition used to prevent, control or suppress asthma or viral infection, the composition comprising a nucleotide molecule capable of interacting with HLA-G.

4. The composition of claim 3 wherein the molecule is a microRNA molecule.

5. A method of identifying a risk factor for asthma, the method comprising:
(a) determining whether an HLA-G allele includes a cytosine or a guanine at position +3142 of HLA-G gene; and
(b) if cytosine is at position +3142, identifying a risk factor for asthma and protection against viral illness in children of mothers with asthma, and
(c) if guanine is at position +3142 identifying a risk factor for asthma and protection against viral illness in children of mothers without asthma.

6. A method for preventing, controlling or suppressing asthma or viral infection, the method comprising:
(a) providing a nucleotide molecule or a microRNA molecule capable of regulating HLA-G; and
(b) preventing, controlling or suppressing asthma or viral infection by regulating HLA-G expression.
